(19) United States
(12) Patent Application Publication
(10) Pub. No.: US 2015/0105624 A1
(43) Pub. Date: Apr. 16, 2015

(54) Surgical access system and related methods

(71) Applicants: Scot Martinelli, Mountain Top, PA (US); Jared Arambula, San Diego, CA (US)

(72) Inventors: Scot Martinelli, Mountain Top, PA (US); Jared Arambula, San Diego, CA (US)

(73) Assignee: NuVasive, Inc., San Diego, CA (US)

(21) Appl. No.: 11/528,996

(22) Filed: Sep. 27, 2006

Related U.S. Application Data
(60) Provisional application No. 60/721,446, filed on Sep. 27, 2005.

(51) Int. Cl. A61B 17/02; A61B 17/34

(52) U.S. Cl. CPC A61B 17/0218; A61B 17/0206; A61B 17/0293; A61B 17/3423; A61B 17/025; A61B 2017/3447; A61B 2017/0256
USPC 600/204

(57) ABSTRACT

A surgical access system and related methods which involve the ability to minimally invasively provide an operative corridor to a disk space while simultaneously providing the ability to distract the disk space. The access system comprises a tissue distraction assembly and a tissue retraction assembly. The tissue distraction assembly (in conjunction with one or more elements of the tissue retraction assembly) is capable of, as an initial step, distracting a region of tissue between the skin of the patient and the surgical target site. The tissue retraction assembly is capable of, as a secondary step, being introduced into this distracted region to thereby define and establish the operative corridor. Once established, any of a variety of surgical instruments, devices, or implants may be passed through and/or manipulated within the operative corridor depending upon the given surgical procedure.

### CROSS REFERENCES TO RELATED APPLICATIONS

The present application is a nonprovisional patent application claiming benefit under 35 U.S.C. §119(e) from U.S. Provisional Application Ser. No. 60/721,446, filed on Sep. 27, 2005, the entire contents of which are hereby expressly incorporated by reference into this disclosure as if set forth fully herein.

### BACKGROUND OF THE INVENTION

I. Field of the Invention

The present invention relates generally to systems and methods for performing surgical procedures and, more particularly, for accessing a surgical target site in order to perform surgical procedures.

II. Discussion of the Prior Art

A noteworthy trend in the medical community is the move away from performing surgery via traditional “open” techniques in favor of minimally invasive or minimal access techniques. Open surgical techniques are generally undesirable in that they typically require large incisions and high amounts of tissue displacement to gain access to the surgical target site, which produces concomitantly high amounts of pain, lengthened hospitalization (increasing health care costs), and high morbidity in the patient population. Less-invasive surgical techniques (including so-called “minimal access” and “minimally invasive” techniques) are gaining favor due to the fact that they involve accessing the surgical target site via incisions of substantially smaller size with greatly reduced tissue displacement requirements. This, in turn, reduces the pain, morbidity and cost associated with such procedures. The access systems developed to date, however, fail in various respects to meet all the needs of the surgeon population.

One drawback associated with prior art surgical access systems relates to the ease with which the operative corridor can be created, as well as maintained over time, depending upon the particular surgical target site. For example, when accessing surgical target sites located beneath or behind musculature or other relatively strong tissue (such as, by way of example only, the psoas muscle adjacent to the spine), or vasculature or other relatively delicate anatomical structures (such as, by way of example only, major blood vessels and/or the esophagus adjacent to the cervical spine), it has been found that advancing an operative corridor-establishing instrument directly through such tissues can be challenging and/or lead to unwanted or undesirable effects (such as stressing or tearing the tissues). While certain efforts have been undertaken to reduce the trauma to tissue while creating an operative corridor, such as (by way of example only) the sequential dilation system of U.S. Pat. No. 5,792,044 to Foley et al., these attempts are nonetheless limited in their applicability based on the relatively narrow operative corridor. More specifically, based on the generally cylindrical nature of the so-called “working cannula,” the degree to which instruments can be manipulated and/or angled within the cannula can be generally limited or restrictive, particularly if the surgical target site is a relatively deep within the patient.

The emerging focus on motion preservation, especially in the cervical spine, as an avenue of treatment for degenerative disk disease only increases the need to be able to establish a suitable operative corridor large enough to effectively treat the disc space and install a motion preservation apparatus without disrupting the nearby anatomic structures. Furthermore, there exists a need to provide the ability to distract the disk space at the same time the operative corridor is established.

The present invention is directed at overcoming, or at least improving upon, the disadvantages of the prior art and need the currently unmet needs of the surgical community.

### SUMMARY OF THE INVENTION

The present invention accomplishes this goal by providing a novel access system and related methods which involve the ability to minimally invasively provide an operative corridor to a disk space while simultaneously providing the ability to distract the disc space. It is expressly noted that, although described herein largely in terms of use in cervical spinal surgery, the access system of the present invention is suitable for use in the lumbar and thoracic areas of the spine, and any number of additional non-spine surgical procedures wherein tissue having significant anatomical structures that must be passed through (or near) in order to establish an operative corridor.

According to one broad aspect of the present invention, the access system comprises a tissue distraction assembly and a tissue retraction assembly. The tissue distraction assembly (in conjunction with one or more elements of the tissue retraction assembly) is capable of, as an initial step, distracting a region of tissue between the skin of the patient and the surgical target site. The tissue retraction assembly is capable of, as a secondary step, being introduced into this distracted region to thereby define and establish the operative corridor. Once established, any of a variety of surgical instruments, devices, or implants may be passed through and/or manipulated within the operative corridor depending upon the given surgical procedure.

The tissue distraction assembly may include any number of components capable of performing the necessary distraction. By way of example only, the tissue distraction assembly may include a K-wire, an initial dilator, and one or more cannulated sequential dilators for performing the necessary tissue distraction to receive the remainder of the tissue retractor assembly thereafter.

The tissue retraction assembly may include any number of components capable of performing the necessary retraction. By way of example only, the tissue retraction assembly may include one or more retractor blades assembled on a ring element. The ring element may include a plurality of ridged regions dimensioned to interact with the retractor blades to maintain the blades in a desired configuration. The retractor blades further may be manipulated to open the retractor assembly; that is, allowing the retractor blades to separate from one another (simultaneously or sequentially) to create an operative corridor to the surgical target site. In a preferred embodiment, this is accomplished by maintaining a pair of lateral retractor blades in a fixed position relative to the cervical surgical target site (so as to avoid having them impinge upon the esophagus or any other significant anatomic structures near the anterior elements of the spine) while the additional retractor blades (i.e. cephalad-most and caudal-most blades) are moved or otherwise translated away from the lateral retractor blades (and each other) so as to create the operative corridor. Optionally, the lateral blades may then be retracted, thus retracting the delicate nearby anatomy. In one optional aspect of the present invention, either the cephalad-most or caudal-most blades, or both, may pivot outward from a central axis of insertion, such that the operative corridor may be expanded. In a further optional aspect of the present invention, the retractor may include a locking element to maintain the blades in an initial alignment during insertion, and a variable-stop mechanism to allow the user to control the degree of expansion of the operative corridor. A blade expander tool may be provided to facilitate manual pivoting of the retractor blades.

The retractor blades may optionally be equipped with a mechanism for transporting or emitting light at or near the surgical target site to aid the surgeon's ability to visualize the surgical target site, instruments and/or implants during the given surgical procedure. According to one embodiment, this mechanism may comprise, but need not be limited to, one or more light sources coupled to the retractor blades such that the terminal ends are capable of emitting light at or near the surgical target site. According to another embodiment, this mechanism may comprise, but need not be limited to, constructing the retractor blades of suitable material (such as clear polycarbonate) and configuration such that light may be transmitted generally distally through the walls of the retractor blade light to shine light at or near the surgical target site. This may be performed by providing the retractor blades having light-transmission characteristics (such as with clear polycarbonate construction) and transmitting the light almost entirely within the walls of the retractor blade (such as by frosting or otherwise rendering opaque portions of the exterior and/or interior) until it exits a portion along the interior (or medially-facing) surface of the retractor blade to shine at or near the surgical target site. The exit portion may be optimally configured such that the light is directed towards the approximate center of the surgical target site and may be provided along the entire inner periphery of the retractor blade or one or more portions therealong.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a perspective view of a tissue retraction assembly forming part of a surgical access system according to one embodiment of the present invention, with the retractor blades in an open or retracted position;

FIG. 2 is a perspective view of the underside of the tissue retraction assembly of FIG. 1;

FIG. 3 is a side view of the tissue retraction assembly of FIG. 1;

FIG. 4 is a top view of the tissue retraction assembly of FIG. 1;

FIG. 5 is a bottom view of the tissue retraction assembly of FIG. 1;

FIG. 6 is a perspective view of the tissue retraction assembly of FIG. 1, with the retractor blades in an initial closed position;

FIG. 7 is a perspective view of the underside of the tissue retraction assembly of FIG. 6;

FIG. 8 is a side view of the tissue retraction assembly of FIG. 6;

FIG. 9 is a top view of the tissue retraction assembly of FIG. 6;

FIG. 10 is a bottom view of the tissue retraction assembly of FIG. 6;

FIG. 11 is a perspective view of a surgical access system according to one embodiment of the present invention, showing in particular the tissue retraction assembly of FIG. 1 inserted over a tissue distraction assembly comprising a series of sequential dilators;

FIG. 12 is a perspective view of the surgical access system of FIG. 11, showing in particular the tissue retraction assembly of FIG. 1 in use with caspar pin insertion dilators; and

FIG. 13 is a perspective view of the surgical access system of FIG. 12, showing in particular the tissue retraction assembly of FIG. 1 in a retracted position in use with caspar pins installed into adjacent vertebrae.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The surgical access system and related methods disclosed herein boasts a variety of inventive features and components that warrant patent protection, both individually and in combination.

The present invention involves accessing a surgical target site in a fashion less invasive than traditional “open” surgeries and doing so in a manner that provides access in spite of the anatomical structures required to be passed through (or near) in order to establish an operative corridor to the surgical target site. Generally speaking, the surgical access system of the present invention accomplishes this by providing a tissue distraction assembly and a tissue retraction assembly.

The tissue distraction assembly of the present invention (comprising an initial dilator and a plurality of sequentially dilating cannulae, with the optional use of a K-wire) is employed to distract the tissues extending between the skin of the patient and a given surgical target site (preferably along the anterior region of the target cervical intervertebral disc). Once distracted, the resulting void or distracted region within the patient is of sufficient size to accommodate a tissue retraction assembly of the present invention. More specifically, the tissue retraction assembly (comprising a plurality of retractor blades extending from a ring element) may be advanced relative to the secondary distraction assembly such that the retractor blades, in a first, closed position, are advanced over the exterior of the secondary distraction assembly. At that point, the retractor blades may be moved into a second, open or “retracted” position to create an operative corridor to the surgical target site. Once the operative corridor is established, any of a variety of surgical instruments, devices, or implants may be passed through and/or manipulated within the operative corridor depending upon the given surgical procedure.

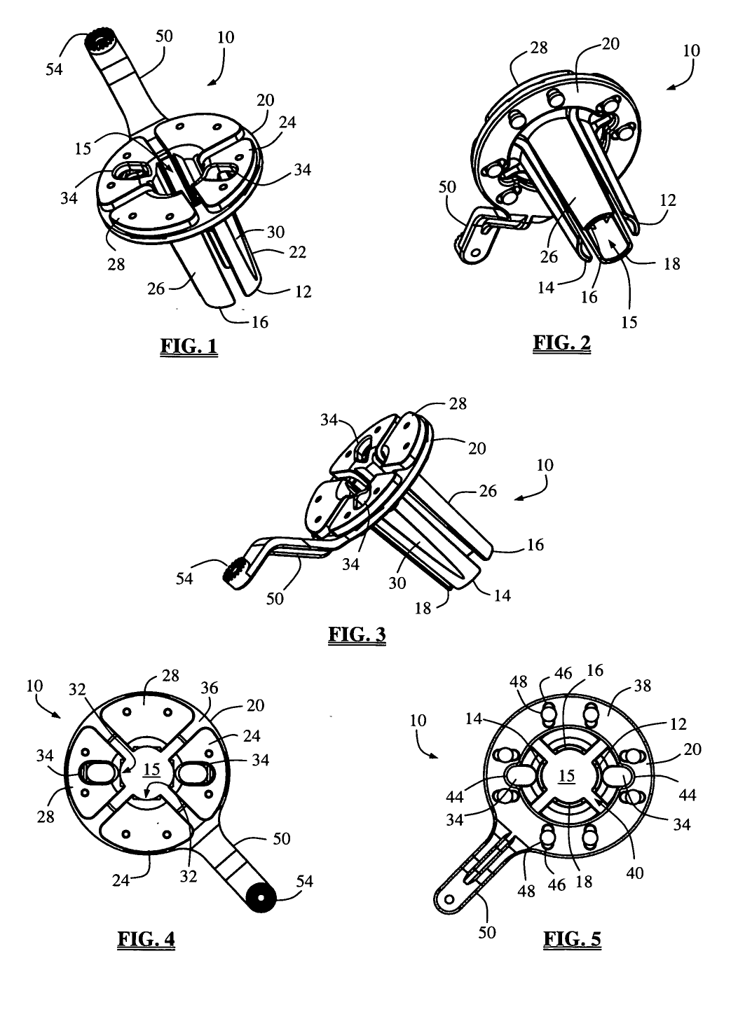

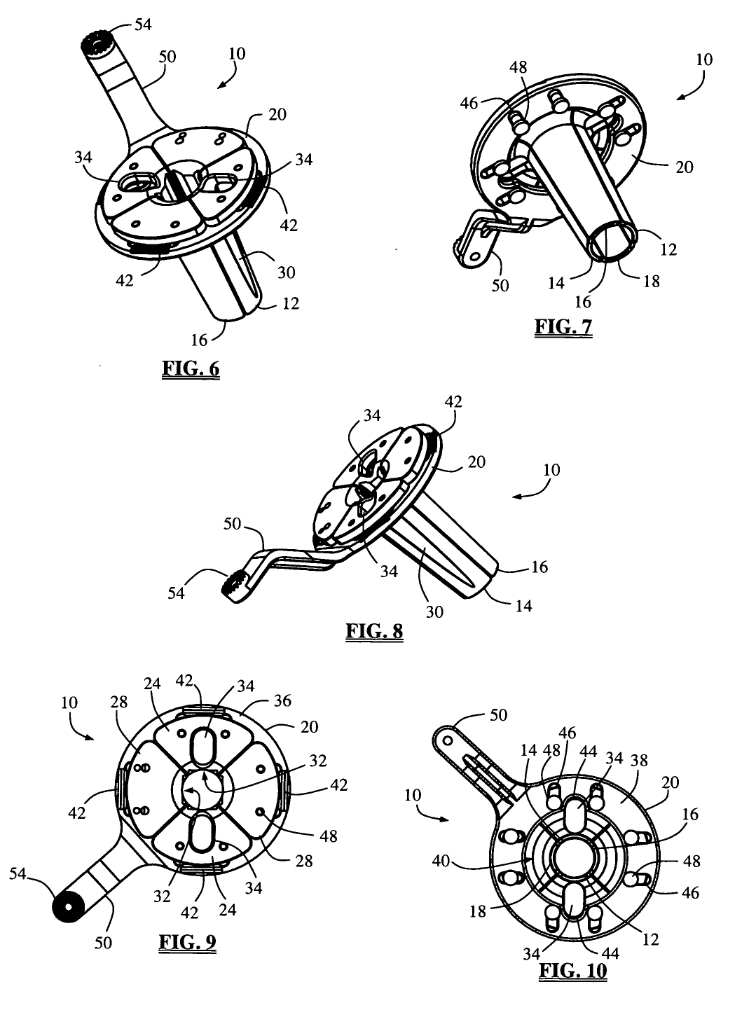

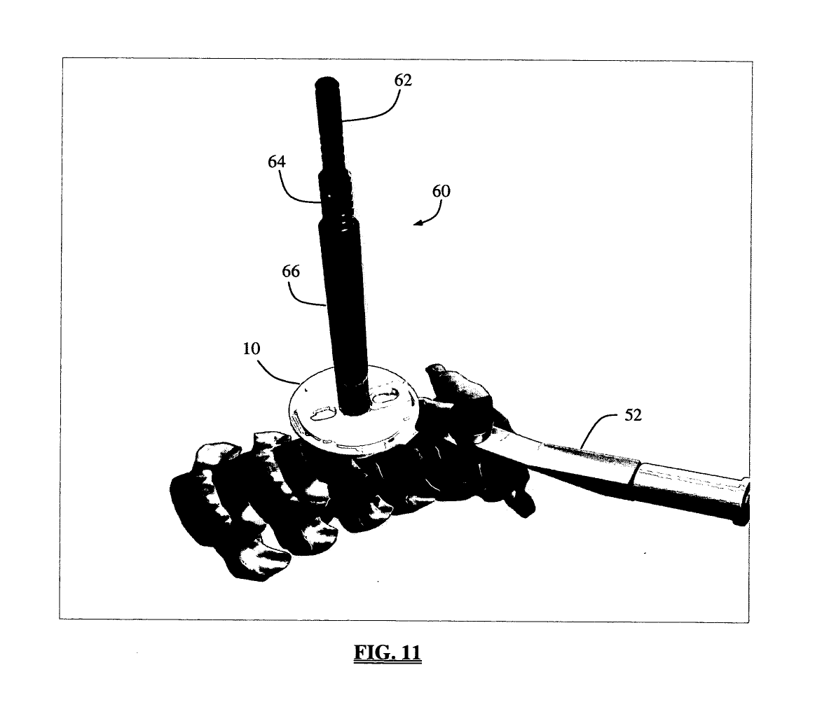

FIGS. 1-10 illustrate a tissue retraction assembly 10 forming part of a surgical access system according to the present invention. The tissue retraction assembly 10 includes a plurality of retractor blades extending from a ring element 20. By way of example only, the ring element 20 is provided with a first retractor blade 12, a second retractor blade 14, a third retractor blade 16, and a fourth retractor blade 18. The retractor assembly 10 is shown in FIGS. 1-5 in a fully retracted or “open” configuration, with the retractor blades 12, 14, 16, 18 positioned a distance from one another so as to form an operative corridor 15 there between and extending to a surgical target site (e.g. an annulus of an intervertebral disc). Although shown and described below with regard to the four-bladed configuration, it is to be readily appreciated that the number of retractor blades may be increased or decreased without departing from the scope of the present invention. Moreover, although described and shown herein with reference to a generally anterior approach to a spinal surgical target site (with the first and second blades 12, 14 being the “cephalad-most” and “caudal-most” blades, respectively, and the third and fourth blades 16, 18 being the lateral blades), it will be appreciated that the retractor assembly 10 of the present invention may find use in any number of different surgical approaches, including generally posterior, generally postero-lateral, generally lateral and generally antero-lateral.

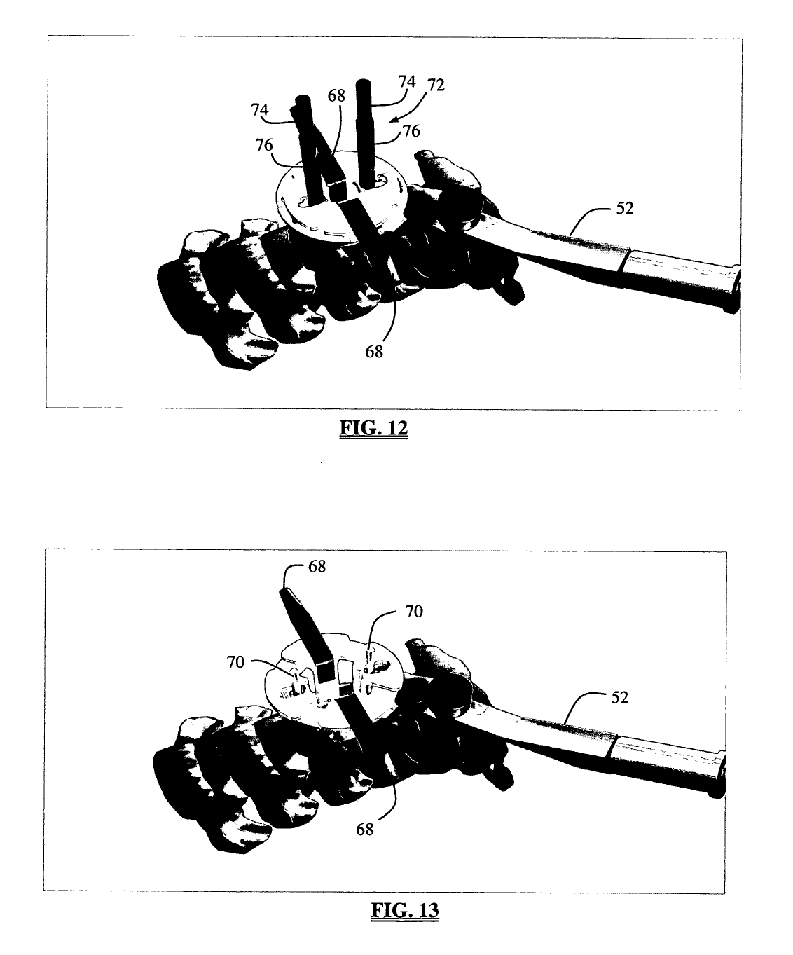

The cephalad-most and caudal-most retractor blades 12, 14 each include an elongated blade region 22 and a proximal attachment region 24. Elongated blade region 22 may be generally curved (or generally flat) such that when the tissue retractor assembly 10 is in a closed position, the retractor blades 12, 14, 16, 18 together have a generally closed geometric configuration corresponding to the geometric shape of the largest sequential dilator. By way of example only, the retractor blades 12, 14, 16, 18 together may have a geometric configuration consisting of generally oval, square, rectangular, polygonal, or circular (shown by way of example in FIG. 10). Elongated blade region 22 may optionally include a depression 30 extending substantially along a radial face and having a generally curved cross-section. The depression 30 is dimensioned to interact with distraction anchors 70 and sequential dilation assembly 72 (described below). The medial face of elongated blade region 22 may include slots 32 or any other suitable attachment mechanisms for receiving a number of utility attachments, including but not limited to shim inserters, retractor extenders, and light-providing elements. Proximal attachment region 24 may be generally flat and generally wedge-shaped such that when the tissue retractor assembly 10 is in a closed position, the proximal attachment regions 24, 28 together generally have a circular configuration (shown by way of example in FIG. 9). Proximal attachment region 24 may further include an elongated aperture 34 dimensioned to receive distraction anchors 70 and sequential dilation assembly 72 (described below).

The lateral retractor blades 16, 18 each include an elongated blade region 26 and a proximal attachment region 28. Elongated blade region 26 may be generally curved (or generally flat) such that when the tissue retractor assembly 10 is in a closed position, the retractor blades 12, 14, 16, 18 together have a generally closed geometric configuration corresponding to the geometric shape of the largest sequential dilator. By way of example only, the retractor blades 12, 14, 16, 18 together may have a geometric configuration consisting of generally oval, square, rectangular, polygonal, or circular (shown by way of example in FIG. 10). The medial face of elongated blade region 26 may include slots 32 or any other suitable attachment mechanisms for receiving a number of utility attachments, including but not limited to shim inserters, retractor extenders, and light-providing elements. Proximal attachment region 28 may be generally flat and generally wedge-shaped such that when the tissue retractor assembly 10 is in a closed position, the proximal attachment regions 24, 28 together generally have a circular configuration (shown by way of example in FIG. 9).

The ring element 20 may be generally circular in shape, and includes superior and inferior surfaces 36, 38 having a central aperture 40 extending therethrough. Superior surface 36 may include a plurality of ridged regions 42 spaced at generally similar intervals around the ring 20. The number of ridged regions 42 may be variable but preferably corresponds to the number of retractor blades provided with the tissue retraction assembly 10. By way of example only, the ring element 20 disclosed herein includes four ridged regions 42 spaced at equidistant intervals around the ring 20, as best shown in FIG. 9. Ridged regions 42 interact with proximal attachment regions 24, 28 to help maintain the retractor blades 12, 14, 16, 18 in an open position after retraction. The ring element 20 includes a plurality of semi-circular cutout regions 44 corresponding to the elongated apertures 34 of the proximal attachment regions 24. The ring element 20 further includes a plurality of elongated pin apertures 46, each dimensioned to receive a connector pin 48. Connector pins 48 are configured to slidably attach the retractor blades 12, 14, 16, 18 to the ring element 20. The elongated pin apertures 46 allow for the pins 48 to slide in a radial or medial direction, depending on whether the retractor blades are being moved to an open (e.g. during retraction) or closed (e.g. prior to removal) position. Each retractor blade may have any number of pins 48 attaching it to ring element 20. By way of example only, the embodiment disclosed herein includes a pair of attachment pins 48 for each retractor blade 12, 14, 16, 18. Correspondingly, the ring element 20 includes eight elongated pin apertures 46 distributed around the ring (best shown in FIGS. 5 & 10).

The ring element 20 further includes an extension 50 for attaching the tissue distraction assembly 10 to an articulating arm 52 (shown by way of example in FIGS. 11-13). Extension 50 may be provided in any length and shape desired to provide rigid attachment to the articulating arm 52 without impacting the accessibility of the operating space. Extension 50 includes a distal connection region 54 configured to attach to articulating arm 52 by attachment means that are commonly known in the surgical arts.

FIG. 11 illustrates a tissue distraction assembly 60 forming part of the surgical access system according to the present invention. The tissue distraction assembly 60 may optionally include a K-wire (not shown), an initial dilating cannula 62, and a plurality of sequentially dilating cannulae 64, 66. In use, the K-wire (if included) is disposed within the initial dilating cannula 62 and the entire assembly 60 is advanced through the tissue towards the surgical target site (i.e. annulus). After the initial dilating cannula 62 is advanced such that the distal end is positioned within the disc space, a first sequential dilating cannula 64 is advanced over initial dilating cannula 62. Optionally, any number of additional sequential dilating cannulae may be employed for the purpose of further dilating the tissue down to the surgical target site. By way of example only, the disclosed embodiment as shown in FIG. 11 includes the further advancement of a second sequential dilating cannula 66 over the cannula 64.

After the tissue has been sufficiently dilated, the tissue retraction assembly 10 of the present invention is thereafter advanced along the exterior of the tissue distraction assembly 60. This is accomplished by maintaining the retractor blades 12, 14, 16, 18 in a first, closed position (with the retractor blades 12-18 in generally abutting relation to one another). Once advanced to the surgical target site, the tissue distraction assembly 60 may be removed. At this point, it may be advantageous for the surgeon to be able to add additional light into the operative corridor 15. For this purpose, a light element 68 may attach to the slots 32 on any of the retractor blades 12, 14, 16, 18. Preferably, the light element 68 is attached to slot 32 on at least one of the lateral retractor blades 16, 18, as shown in FIG. 12. Optionally, the light element 68 may be molded into or otherwise provided within the retractor blades 12-18 such that an additional attachment is not necessary.

The surgeon may find it advantageous to distract the disc space prior to or simultaneously with retracting the tissue. For this purpose, it may be necessary to install distraction anchors 70 into each vertebra adjacent the target disc space. FIG. 12 illustrates a sequential dilation assembly 72 for use in the insertion of the distraction anchors 70. Sequential dilation assembly 72 functions in a similar manner to tissue dilation assembly 60 described above. An initial dilator 74 is advanced through elongated slots 34 on proximal attachment regions 24 of first and second retractor blades 12, 14. Following this initial distraction, any number of cannulated secondary distraction dilators may be used to distract the tissue as desired. By way of example only, the present embodiment is shown using one subsequent distraction dilator 76. Once the distraction dilator 76 has been inserted, the initial dilator 74 may be removed and the distraction anchors 70 may be inserted into the vertebra through the subsequent distraction dilator 76. Distraction anchors 70 may consist of any device capable of providing purchase to the bone to allow for the distraction of the disc space. By way of example only, the distraction anchors may include caspar pins (as shown in FIG. 13). Once the distraction anchors 70 have been inserted into the vertebrae, the distraction dilators 76 may be removed.

At this point, a disc distraction assembly (not shown) may engage with the distraction anchors 70 and be operated to distract the disc space. Optionally, the tissue retraction assembly 10 may be configured to attach to the disc distraction assembly and/or the distraction anchors 70 to simultaneously move the first and second retractor blades 12, 14 into a second, open or “retracted” position as shown generally in FIGS. 1-5 and 13. Preferably, the lateral retractor blades 16, 18 are allowed to stay in the same general position during this process, such that the caudal-most and cephalad-most retractor blades 12, 14 move away from the lateral retractor blades 16, 18. At this point, lateral retractor blades 16, 18 may be optionally retracted to move nearby delicate anatomy (e.g. esophagus, blood vessels) away from the operative corridor 15. In the alternative, tissue retraction assembly 10 may be configured such that the lateral retractor blades 16, 18 retract simultaneously with the caudal-most and cephalad-most retractor blades 12, 14. At this point, the operative corridor 15 is established and the surgeon can insert any instruments and/or devices as desired.

The tissue retraction assembly 10 of the present invention may be composed of any material suitable to provide an operative corridor, including but not limited to titanium, metal, ceramics, plastic, or any combination of titanium, metal, ceramic, and plastic.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the invention is to cover all modifications, equivalents, and alternatives falling within the spirit and scope of the invention as defined herein. For example, while the invention is described herein with a specific focus on application to the cervical spine, it is to be understood that the surgical access system of the present invention may be useful in providing minimally disruptive surgical access corridors to many different parts of the body.

1. A surgical retractor for creating an operative corridor to a surgical target site, comprising:
a body portion having a first surface, a second surface opposite said first surface, and a central aperture extending therethrough; and
at least two retractor blades moveably attached to said body portion, each retractor blade including an attachment portion configured to interact with said first surface, and a blade portion extending generally perpendicularly from said attachment portion through said central aperture, wherein a first of said at least two retractor blades moves independently of a second of said at least two retractor blades, such that said at least two retractor blades are advanced simultaneously to said surgical target site while in a closed position and thereafter selectively opened to create a customized operative corridor to said surgical target site.

2. The surgical retractor of claim 1, wherein said body portion has a generally circular shape.

3. The surgical retractor of claim 1, wherein said attachment portion is adapted to migrate along said first surface away from a center of said central aperture.

4. The surgical retractor of claim 1, wherein said first surface includes at least one anti-migration element configured to interact with said attachment portion of said retractor blade to limit uncontrolled movement of said retractor blade.

5. The surgical retractor of claim 4, wherein said anti-migration element comprises a ridged region.

6. The surgical retractor of claim 1, wherein said attachment portion includes at least one aperture for receiving an attachment member configured to couple said attachment portion to said body.

7. The surgical retractor of claim 1, wherein said body portion includes a plurality of second apertures, one second aperture for each of said retractor blades, said second apertures each configured to receive an attachment member adapted to couple said attachment portion to said body.

8. The surgical retractor of claim 7, wherein said each of said second apertures comprises an elongated slot to allow for migration of said attachment member.

9. The surgical retractor of claim 7, wherein said body portion further includes a plurality of third apertures, one third aperture for each of said retractor blades, said third apertures each configured to receive an attachment member adapted to couple said attachment portion to said body portion.

10. The surgical retractor of claim 9, wherein each of said third apertures comprises and elongated slot to allow for migration of said attachment member.

11. The surgical retractor of claim 7, wherein said attachment member comprises a pin.

12. (canceled)

13. A system for accessing a surgical target site, comprising:
a retraction assembly including a body portion and at least two retractor blades moveably attached to said body portion, said body portion having a first surface, a second surface opposite said first surface, and a central aperture extending therethrough, said retractor blades each having an attachment portion configured to interact with said first surface, and a blade portion extending generally perpendicularly from said attachment portion through said aperture, wherein a first of said at least two retractor blades moves independently of a second of said at least two retractor blades; and
a distraction assembly for creating a distraction corridor to a surgical target site prior to advancing said retractor blades to said surgical target site.

14. The system of claim 13, wherein said body portion of said retraction assembly has a generally circular shape.

15. The system of claim 13, wherein said attachment portions of said retractor blades are adapted to migrate along said first surface away from a center of said central aperture.

16. The system of claim 13, wherein said first surface of said body portion of said retraction assembly includes at least one anti-migration element configured to interact with said attachment portion of said retractor blade to limit uncontrolled movement of said retractor blade.

17. The system of claim 16, wherein said anti-migration element comprises a ridged region.

18. The system of claim 13, wherein said attachment portion includes at least one aperture for receiving an attachment member configured to couple said attachment portion to said body.

19. The system of claim 13, wherein said body portion includes a plurality of second apertures, one second aperture for each of said retractor blades, said second apertures each configured to receive an attachment member adapted to couple said attachment portion to said body portion.

20. The system of claim 19, wherein said each of said second apertures comprises an elongated slot to allow for migration of said attachment member.

21. The system of claim 19, wherein said body portion further includes a plurality of third apertures, one third aperture for each of said retractor blades, said third apertures each configured to receive an attachment member adapted to couple said attachment portion to said body.

22. The system of claim 21, wherein each of said third apertures comprises and elongated slot to allow for migration of said attachment member.

23. The system of claim 19, wherein said attachment member comprises a pin.

24. (canceled)

25. The system of claim 13, wherein said distraction assembly includes a K-wire and at least one dilator capable of being slidably passed over said K-wire to perform said initial distraction.

26. A method of establishing an operative corridor to a surgical target site, comprising:
providing an initial distraction assembly to create a distraction corridor to said surgical target site;
simultaneously advancing at least two retractor blades along said distraction corridor to said surgical target site while in a first position, said at least two blades forming part of a retraction assembly further including a body portion having a first surface, a second surface opposite said first surface, and a central aperture configured to receive said at least two retractor blades, wherein each retractor blade includes an attachment portion configured to interact with said first surface, and a blade portion configured to extend through said central aperture;
moving a first of said at least two retractor blades from a first position to a second position independently of a second retractor blade to establish said operative corridor to said surgical target site; and
moving said second of said at least two retractor blades from a first position to a second position to retract adjacent anatomy away from said operative corridor.

27. The method of claim 26, wherein said initial distraction assembly includes a K-wire and at least one dilator capable of being slidably passed over said K-wire to perform said initial distraction.

28. The method of claim 26, wherein said body portion of said retraction assembly has a generally circular shape.

29. The method of claim 26, wherein each of said at least two retractor blades includes an attachment portion configured to interact with said first surface and a blade portion extending from said attachment portion, said attachment portion adapted to migrate along said first surface away from a center of said central aperture.

30. The method of claim 29, wherein said first surface of said body portion of said retraction assembly includes at least one anti-migration element configured to interact with said attachment portion of said retractor blade to limit uncontrolled movement of said retractor blade.

31. The method of claim 30, wherein said anti-migration element comprises a ridged region.

32. The method of claim 29, wherein said attachment portion includes at least one aperture for receiving an attachment member configured to couple said attachment portion to said body portion.

33. The method of claim 29, wherein said body portion includes a plurality of second apertures, one second aperture for each of said retractor blades, said second apertures each configured to receive an attachment member adapted to couple said attachment portion to said body.

34. The method of claim 33, wherein said each of said second apertures comprises an elongated slot to allow for migration of said attachment member.

35. The method of claim 33, wherein said body portion further includes a plurality of third apertures, one third aperture for each of said retractor blades, said third apertures each configured to receive an attachment member adapted to couple said attachment portion to said body.

36. The method of claim 35, wherein each of said third apertures comprises and elongated slot to allow for migration of said attachment member.

37. The method of claim 33, wherein said attachment member comprises a pin.

38. (canceled)

39. The surgical retractor of claim 1, further comprising a third retractor blade positioned opposite said first retractor blade and a fourth retractor blade positioned opposite said second retractor blade.

40. The surgical retractor of claim 39, wherein said first and third retractor blades simultaneously move from a first position to a second position.

41. The surgical retractor of claim 39, wherein said first and third retractor blades move independently of said second and fourth retractor blades.
